(19) United States
(12) Patent Application Publication
(10) Pub. No.: US 2023/0414902 A1
(43) Pub. Date: Dec. 28, 2023

(54) SUPPORT CATHETER WITH ENHANCED TORQUE RESPONSE AND HIGH PUSHABILITY

(71) Applicants: Boston Scientific Medical Device Limited, Galway (IE); Boston Scientific Scimed, Inc., Maple Grove, MN (US)

(72) Inventors: SUMIT MALIK, Gurgaon (IN); ABHISHEK BINDRA, Rohtak (IN); JOEL M. WASDYKE, Independence, MN (US); MAHENDRA PRATAP SINGH, Gurgaon (IN)

(73) Assignees: Boston Scientific Medical Device Limited, Galway (IE); Boston Scientific Scimed, Inc., Maple Grove, MN (US)

(21) Appl. No.: 18/212,884

(22) Filed: Jun. 22, 2023

Related U.S. Application Data
(60) Provisional application No. 63/354,732, filed on Jun. 23, 2022.

(51) Int. Cl. A61M 25/01; A61M 25/00; A61M 25/09

(52) U.S. Cl. CPC A61M 25/0144; A61M 25/0108; A61M 25/0045; A61M 25/09

(57) ABSTRACT

A support catheter may be used in combination with a guidewire or other medical device. The support catheter may be adapted to provide both high pushability and good torque response. The elongate shaft of the support catheter may include a polymeric inner layer, stainless steel braided layer disposed over the polymeric inner layer, and a polymeric blend layer that is extruded directly over the stainless-steel braided layer. The construction of the elongate shaft may provide the support catheter with a pushability value of at least 900 grams per centimeter and a torque response value of less than 300 degrees.

### CROSS-REFERENCE TO RELATED APPLICATIONS

This application claims the benefit of U.S. Prov. Pat. App. No. 63/354,732, filed Jun. 23, 2022, titled SUPPORT CATHETER WITH ENHANCED TORQUE RESPONSE AND HIGH PUSHABILITY, which is incorporated herein by reference.

### TECHNICAL FIELD

The present disclosure pertains to medical devices. More particularly, the disclosure is directed to medical devices such as support catheters that provide both good torque response and high pushability.

### BACKGROUND

A wide variety of medical devices have been developed for medical use, for example, for use in accessing body cavities and interacting with fluids and structures in body cavities. For example, catheters may provide access to remote locations within the vasculature. Such catheters may be able to provide support to other medical devices, a flow path for fluid delivery to a remote target site, or means for performing other therapeutic and/or diagnostic actions.

### SUMMARY

This disclosure provides design, material, manufacturing method, and use alternatives for medical devices. A support catheter may be used in combination with a guidewire for additional support when attempting to cross a CTO (chronic total occlusion) when treating peripheral artery disease. The support catheter may be adapted to provide both high pushability and good torque response. The support catheter may include a polymeric inner layer, a braided layer (such as stainless-steel braided filaments) disposed over the polymeric inner layer, and a polymeric outer layer, such as a polymeric blend layer that is extruded directly over the braided layer. As a result, the support catheter may exhibit both good pushability and good torque response.

One example is a support catheter comprising an elongate shaft extending from a proximal region to a distal region. The elongate shaft includes a polymeric inner layer extending from the proximal region to the distal region, a stainless-steel braided layer exterior to the polymeric inner layer and extending from the proximal region to the distal region, and a polymeric blend outer layer partially embedded within the stainless-steel braided layer. The polymeric blend outer layer if formed of a blend of polyether block amide (PEBA) polymers. The elongate shaft is adapted to provide a pushability value of at least 900 grams per centimeter and a torque response value of less than 300 degrees.

Alternatively or additionally, the pushability value may be at least 1000 grams per centimeter.

Alternatively or additionally, the torque response value may be less than 200 degrees.

Alternatively or additionally, the torque response value may be less than 100 degrees.

Alternatively or additionally, the support catheter further comprises a distal tip forming a butt joint with a distal end of the elongate shaft.

Alternatively or additionally, the braided layer extends along an entire length of the elongate shaft to the butt joint.

Alternatively or additionally, the support catheter further comprises a first radiopaque marker band located proximal of the butt joint and a polymeric sleeve spanning the butt joint. The polymeric sleeve extends over and surrounds the first radiopaque marker band and the distal tip.

Alternatively or additionally, the support catheter further comprises a second radiopaque marker band proximal of and spaced apart from the first radiopaque marker band. The outer layer is reflowed around the second radiopaque marker band such that the second radiopaque marker band is embedded into the outer layer with the outer layer in contact with a proximal annular surface and a distal annular surface of the second radiopaque marker band.

Alternatively or additionally, an outer diameter of the second radiopaque marker band is greater than an outer diameter of the outer layer just proximal and distal of the second radiopaque marker band.

Alternatively or additionally, the polymeric blend outer layer comprises a blend of VESTAMID® Care ME71 and VESTAMID® Care ML 18.

Alternatively or additionally, the support catheter comprises one of a large support catheter adapted to accommodate a 0.035-inch guidewire, a medium support catheter adapted to accommodate a 0.018-inch guidewire, and a small support structure adapted to accommodate a 0.014-inch guidewire, wherein the large support catheter is adapted to allow either the medium support catheter or the small support catheter to be advanced within the large support catheter.

Alternatively or additionally, the polymeric inner layer comprises a bi-layer structure with a perfluoro inner layer and a PEBA outer layer.

Another example is a support catheter. The support catheter includes an elongate shaft having a proximal end and a distal end. The elongate shaft includes a perfluoro inner layer extending continuously from the proximal end to the distal end, a stainless-steel braided layer exterior to the perfluoro inner layer and extending continuously from the proximal end to the distal end, and a polymeric outer layer extending continuously from the proximal end to the distal end. The polymeric outer layer surrounds and is partially embedded within the stainless-steel braided layer. The polymeric outer layer comprises a blend of VESTAMID® Care PEBA polymers. A distal tip is joined to the elongate shaft at a butt joint at the distal end of the elongate shaft. The elongate shaft is adapted to provide a pushability value of at least 900 grams per centimeter and a torque response value of less than 300 degrees.

Alternatively or additionally, the pushability value may be at least 1000 grams per centimeter.

Alternatively or additionally, the torque response value may be less than 200 degrees.

Alternatively or additionally, the torque response value may be less than 100 degrees.

Alternatively or additionally, the support catheter comprises a polymeric tie layer disposed between the perfluoro inner layer and the stainless-steel braided layer.

Alternatively or additionally, the support catheter comprises a first radiopaque marker band located proximal of the butt joint and a polymeric sleeve spanning the butt joint. The polymeric sleeve extends over and surrounds the first radiopaque marker band and the distal tip.

Alternatively or additionally, the support catheter is packaged in a linear orientation in order to avoid providing a curved set to the support catheter.

Alternatively or additionally, the support catheter comprises one of a large support catheter adapted to accommodate a 0.035-inch guidewire, a medium support catheter adapted to accommodate a 0.018-inch guidewire, and a small support catheter adapted to accommodate a 0.014-inch guidewire.

Alternatively or additionally, the outer layer comprises a blend of VESTAMID® Care ME71 and VESTAMID® Care ML 18, and wherein the large support catheter has a polymeric blend outer layer comprising VESTAMID® Care ME71 and VESTAMID® Care ML 18 blended in a first ratio, the medium support catheter has a polymeric blend outer layer comprising VESTAMID® Care ME71 and VESTAMID® Care ML 18 blended in a second ratio, and the small support catheter has a polymeric blend outer layer comprising VESTAMID® Care ME71 and VESTAMID® Care ML 18 blended in a third ratio.

Alternatively or additionally, the large support catheter is adapted to allow either one of the medium support catheter and the small support catheter to be advanced within the large support catheter.

Another example is a support catheter including tan elongate shaft extending from a proximal end to a distal end. The elongate shaft includes a polymeric inner layer extending continuously from the proximal end to the distal end, a braided layer surrounding the polymeric inner layer and extending continuously from the proximal end to the distal end, and a polymeric blend outer layer extending continuously from the proximal end to the distal end. The polymeric blend outer layer surrounds and is partially embedded within the braided layer. The polymeric blend outer layer comprises a blend of polyether block amide (PEBA) polymers. A distal tip is secured to the distal end of the elongate shaft via a butt joint. A plurality of radiopaque marker bands are disposed within the distal region proximal of the butt joint. The support catheter is adapted to provide a pushability value of at least 900 grams per centimeter and a torque response value of less than 300 degrees.

Alternatively or additionally, the pushability value may be at least 1000 grams per centimeter.

Alternatively or additionally, the torque response value may be less than 200 degrees.

Alternatively or additionally, the torque response value may be less than 100 degrees.

Alternatively or additionally, the support catheter comprises a polymeric sleeve spanning the butt joint. The polymeric sleeve extends over and surrounds a distalmost one of the plurality of radiopaque marker bands and the distal tip.

Alternatively or additionally, the plurality of radiopaque marker bands includes a proximalmost one of the plurality of radiopaque marker bands located proximal of the distalmost one of the plurality of radiopaque marker bands. The polymeric blend outer layer is reflowed around the proximalmost one of the plurality of radiopaque marker bands such that the proximalmost one of the plurality of radiopaque marker bands is embedded into the polymeric blend outer layer with the polymeric blend outer layer in contact with a proximal annular surface and a distal annular surface of the proximalmost one of the plurality of radiopaque marker bands.

Alternatively or additionally, the polymeric blend outer layer comprises a blend of VESTAMID® Care ME71 and VESTAMID® Care ML 18.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side view of an illustrative support catheter;

FIG. 2 is an enlarged cross-sectional view of a portion of the illustrative support catheter of FIG. 1;

FIG. 3 is an enlarged view of a portion of the illustrative support catheter of FIG. 1;

FIG. 4 is a partially cutaway view of a portion of the elongate shaft forming part of the illustrative support catheter of FIG. 1;

FIG. 5 is a side view of a portion of a braided layer forming part of the illustrative support catheter of FIG. 1;

FIG. 6 is a schematic view of an assembly that includes a packaging structure and the illustrative support catheter of FIG. 1 disposed within the packaging structure;

FIG. 7 is a side view of a prior art support catheter;

FIGS. 7A and 7B are cross-sectional views of the prior art support catheter of FIG. 7;

FIG. 8 is a side view of a prior art support catheter;

FIGS. 8A, 8B, 8C and 8D are cross-sectional views of the prior art support catheter of FIG. 8;

FIG. 9 is a graph providing experimental results; and

FIG. 10 is a graph providing experimental results.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

In some instances, a guidewire may be used in order to cross a CTO (chronic total occlusion) when treating peripheral arterial disease. This may include directing the guidewire towards a lesion at an ostium of an artery without entering the collaterals. The guidewire may be directed towards a particular vessel having a sharp take-off angle for access. A physician or other professional may desire to find a soft spot on the lesion cap to make it easier for the guidewire to cross the lesion. In some instances, the guidewire may be directed to help the guidewire re-enter the lumen in a sub-intimal crossing technique.

In some instances, a physician or other professional may wish to use a support catheter with the guidewire in order to provide additional support to the guidewire. During the procedure, the support catheter may be torqued in order to help direct the guidewire into the intended vessel as well as finding the soft spot on the lesion cap for crossing. Accordingly, a highly controllable support catheter that can provide a 1:1 torque response is helpful. Having high pushability is also desired. Many catheters have good torque response. Many catheters have good pushability. However, these catheters are generally a trade-off between these competing properties, and do not provide a combination of good torque response and good pushability in a single catheter.

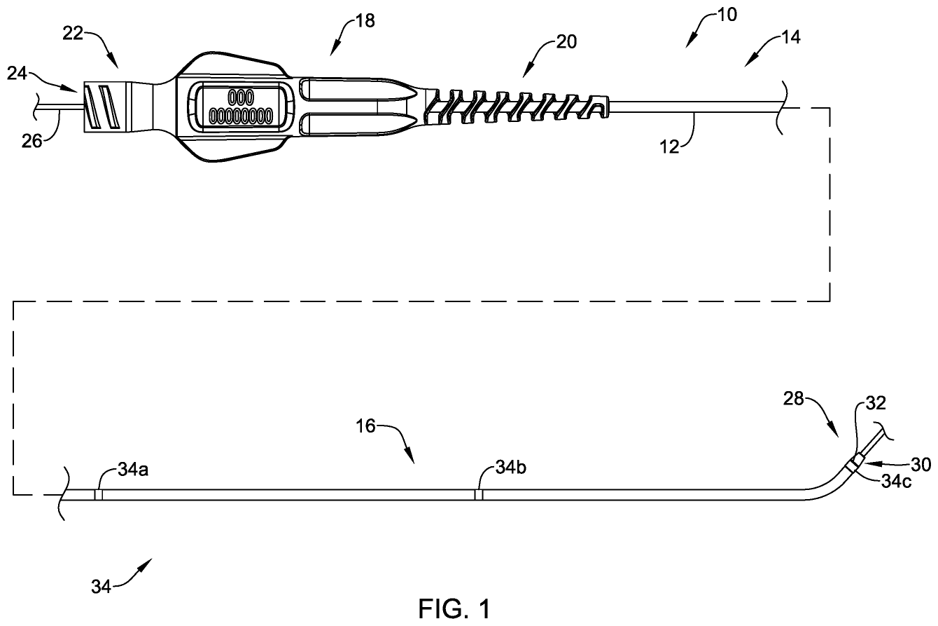

FIG. 1 is a side view of an illustrative support catheter 10. The illustrative support catheter 10 is, as will be discussed, adapted to provide both good torque response and good pushability. Accordingly, the support catheter 10 may be considered as being well-equipped to be used with a guidewire in crossing a CTO (chronic total occlusion), for example. In some cases, the support catheter 10 provides a pushability value of at least 900 grams per centimeter (g/cm). In some cases, the support catheter 10 provides a pushability value of at least 1000 g/cm. The support catheter 10 may also provide a torque response value (lag) of less than 300 degrees. In some cases, the support catheter 10 may provide a torque response value of less than 200 degrees. In some cases, the support catheter 10 may provide a torque response value of less than 100 degrees. The tests used to ascertain pushability value and torque response value are described below in the experimental section.

The support catheter 10 includes an elongate shaft 12 that extends from a proximal region 14 to a distal region 16. The proximal region 14 includes A catheter hub 18 with a strain relief 20 is fixedly attached to the proximal region 14 with the elongate shaft 12 extending distally therefrom. In some cases, the strain relief 20 may be integrally molded as part of the catheter hub 18. The catheter hub 18 also includes a Luer fitting 22 that is adapted to allow various devices, such as fluid lines, to be fluidly coupled with the catheter hub 18. The catheter hub 18 defines a lumen 24 that extends through the catheter hub 18. In some cases, the lumen 24 aligns with a lumen (not shown in FIG. 1) that extends through the elongate shaft 12. The lumen of the elongate shaft 12 may extend to the distal end of the elongate shaft 12. The lumen 24 (and the aforementioned lumen extending through the elongate shaft 12) is adapted to accommodate a guidewire 26 that can be seen entering the catheter hub 18 and exiting at a distal end 28 of the elongate shaft 12.

The distal region 16 of the elongate shaft 12 includes a distal tip 30 that joins the elongate shaft 12 at a butt joint 32. In some instances, the distal region 16 may be linear such that the central axis through the distal tip 30 is coaxial with the central axis of the proximal region 14 of the elongate shaft 12. In some cases, as shown, the distal region may have a curved profile to help with steerability of the support catheter 10. In some an instance, the central axis of the distal tip 30 extends at an acute angle to the central axis of the proximal region 14 proximal of the bend in the distal region 16. In some instances, the acute angle may be about 20 degrees, about 40 degrees, about 45 degrees, or about 48 degrees, for example. In some cases, the support catheter 10 includes a plurality of marker bands 34, such as three spaced apart marker bands labeled individually as 34a, 34b and 34c. The marker bands 34 may be formed of any radiopaque material, such as tungsten, platinum, or gold. The marker bands 34 may be formed having any desired length, and any desired spacing between the individual marker bands 34. In some cases, each of the marker bands 34 may have a length of 1 mm and may have a thickness of 0.001 inches. The marker bands 34 may each be spaced 5 centimeters (cm) apart along the distal region 16 of the elongate shaft. The distalmost marker band 34c may be located just proximal of the butt joint 32.

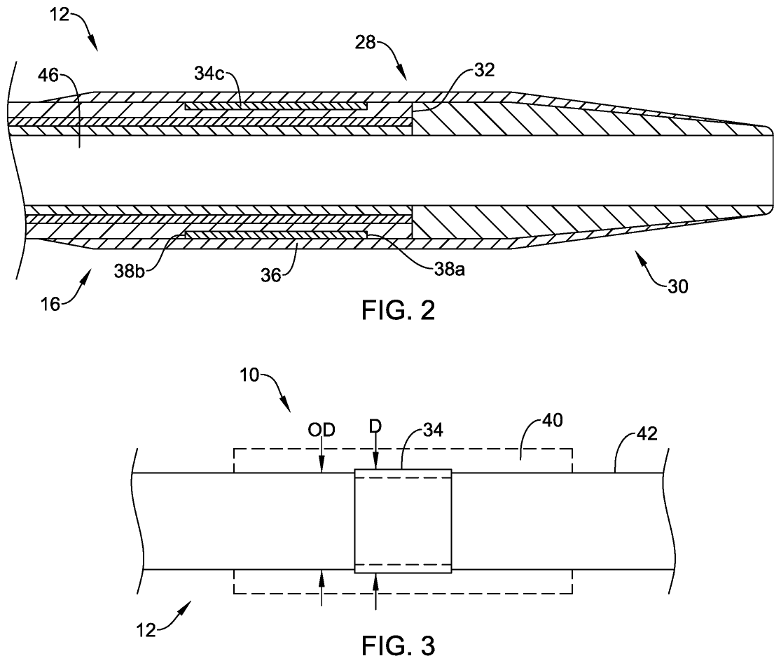

In some cases, one or more of the marker bands 34 may undergo edge smoothing, or swaging. This is a process in which a heat shrink polymer tubing is placed over the marker band 34, and sufficient heat is applied to soften the underlying polymer layer of the elongate shaft 12 to cause the marker band 34 to push at least partially into the underlying polymer layer of the elongate shaft 12 through the radially inward force of the heat shrink polymer tubing, thereby reducing the profile of the marker band 34. In some cases, a polymeric sheath may extend over the butt joint 32 and the marker band 34c to reinforce the butt joint 32. FIG. 2 is an enlarged view of a portion of FIG. 1, showing a polymeric sheath 36 that extends over the butt joint 32. The polymeric sheath 36 may extend proximal of the butt joint 32 and surround a portion of the distal region 16 of the elongate shaft 12 (e.g., the distal extent of the elongate shaft 12). Further, the polymeric sheath 36 may extend distal of the butt joint 32 and surround the distal tip 30. In some cases, the polymeric sheath 36 may also extend over the distalmost marker band 34c. In some cases, this can help to encapsulate the distal and proximal edges 38a and 38b of the marker band 34c and thus protect against accidental tissue damage and/or catching on another medical device that could otherwise be caused by the edges 38a and 38b. The polymeric sheath 36 may be formed of any suitable polymer. In some cases, the polymeric sheath 36 may be formed of a PEBAX® material such as but not limited to PEBAX® 72D, or perhaps a Vestamid® material.

In some cases, the marker bands 34 that are not covered by the polymeric sheath 36, such as the proximalmost marker band 34a and the intermediate marker band 34b, may be edge smoothed. Edge smoothing is a process in which a heat shrink polymer tubing is disposed over the marker band 34 and sufficient heat is applied to soften the underlying polymer layer of the elongate shaft 12 to cause the marker band 34 to push at least partially into the softened material of the elongate shaft 12 through the radially inward force of the marker band 34 being swaged or crimped onto the elongate shaft 12. In some cases, this may cause partial reflow of the outer layer of the elongate shaft 12 around the distal edge 38a and/or the proximal edge 38b of the marker band 34, also helping to reduce the overall radial profile of the marker band 34 relative to an outer surface of the elongate shaft 12.

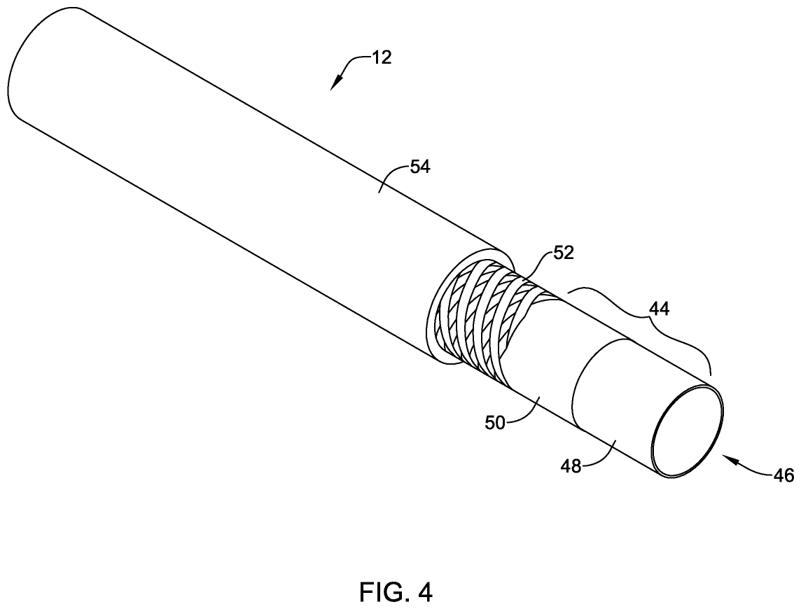

FIG. 3 shows an enlarged view of a portion of FIG. 1, showing a heat shrink polymer segment 40 (in dashed lines) extending across the marker band 34. In this view, the heat shrink polymer segment 40 has already been heat-shrunk, which helps to partially reflow the outer surface 42 of the elongate shaft 12 and thus reduce the overall radial profile of the marker band 34 relative to the outer surface 42 of the elongate shaft 12. For example, in one instance the outer diameter D of the marker band 34 may be about 0.0553 to 0.0576 inches, whereas the outer diameter OD of the elongate shaft 12 of the support catheter (i.e., the large support catheter), may be a constant diameter along an entire length of the elongate shaft (except for underneath the marker band 34) of about 0.0545±0.002 inches, for example. In another instance, the outer diameter D of the marker band 34 may be about to 0.0362 inches, whereas the outer diameter OD of the elongate shaft 12 of the support catheter (i.e., the medium support catheter), may be a constant diameter along an entire length of the elongate shaft (except for underneath the marker band 34) of about ±0.002 inches, for example. In yet another instance, the outer diameter D of the marker band 34 may be about 0.0321 to 0.0341 inches, whereas the outer diameter OD of the elongate shaft 12 of the support catheter (i.e., the small support catheter), may be a constant diameter along an entire length of the elongate shaft (except for underneath the marker band 34) of about 0.0310±0.002 inches, for example. Likewise, the inner diameter of the marker band 34 after being swaged, crimped, or otherwise secured around the outer layer of the elongate shaft 12 may be less than the outer diameter OD of the outer layer of the elongate shaft 12 just proximal and distal of the marker band 34.

In some cases, the heat shrink polymer segment 40 may subsequently be removed from the support catheter 10, leaving the circumferential outer surface of the maker band 34 exposed and uncovered from the elongate shaft 12. In some instances, it is contemplated that the heat shrink polymer segment 40 may remain as part of the support catheter 10 extending over the outer surface of the marker band 34 and extending proximally and distally beyond the marker band 34. When the outer layer of the elongate shaft 12 is reflowed around the proximal and distal edges of the marker band 34, the polymer material of the outer layer of the elongate shaft 12 may contact and substantially cover the proximal and distal annular faces of the marker band 34 except for the portion radially beyond the outer diameter OD of the elongate shaft 12. In other words, the outer diameter OD of the elongate shaft 12 just proximal and distal of the marker band 34 (i.e., at the proximal and distal edges of the marker band 34) may be greater than the inner diameter of the marker band 34 such that the outer layer of the elongate shaft 12 is juxtaposed with the proximal and distal annular faces of the marker band 34.

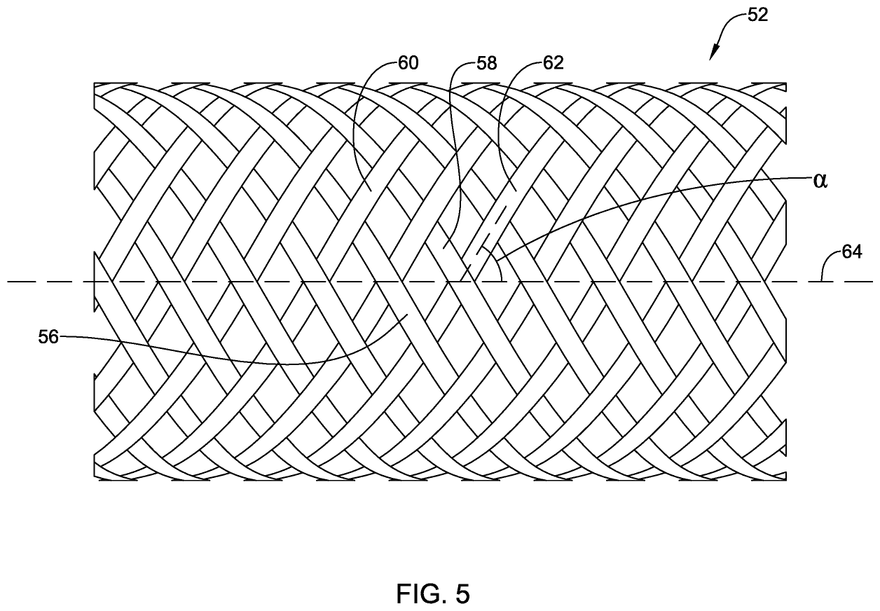

FIG. 4 is a partially cut-away view of the elongate shaft 12. This cut-away view may be taken anywhere along the elongate shaft 12, as in some instances the elongate shaft 12 has the same construction all along the entire length of the elongate shaft 12, extending between the catheter hub 18 and the butt joint 32 joining the distal tip 30 to the elongate shaft 12. In some cases, the elongate shaft 12 has the same dimensions, such as a constant inner diameter (ID) and a constant outer diameter (OD) along the entire length of the elongate shaft 12 from the catheter hub 18 to the butt joint 32, and thus the same wall thicknesses of each of the layers forming the elongate shaft 12, all along the length of the elongate shaft 12.

In some cases, the support catheter 10 may be made in multiple sizes (e.g., outer diameters), which for sake of discussion may be referred to as a large support catheter, a medium support catheter and a small support catheter. In some cases, the large support catheter may be adapted to have a lumen large enough allow either the medium support catheter or the small support catheter to extend within the large support catheter. In other words, the diameter of the lumen of the large support catheter may be greater than the outer diameter of both the medium support catheter and the small support catheter. This may be facilitated by each of the sizes of the support catheter 10 having constant dimensions (e.g., constant outer diameter and/or constant inner diameter) along the length of the elongate shaft 12 forming each of the sizes of the support catheter 10, and thus not having any tapers along the length of the elongate shaft 12 (except for the tapered outer diameter of the distal tip at the extreme distal end of the support catheter). In some cases, for example, a physician or other professional may be using a small support catheter or a medium support catheter to provide support to a guidewire such as the guidewire 26 in attempting to cross an occlusion, and may decide that additional support would be helpful. In such a case, the physician or other professional may decide to temporarily withdraw the small support catheter or the medium support catheter from the guidewire, advance a large support catheter over the guidewire, and then advance the previously used small support catheter over the guidewire and through the lumen of the large support catheter. The outer diameter of the large support catheter, which may be a constant diameter along an entire length of the elongate shaft, may be about 0.0545±0.002 inches, for example. The outer diameter of the medium support catheter, which may be a constant diameter along an entire length of the elongate shaft, may be about 0.0335±0.002 inches, for example. The outer diameter of the small support catheter, which may be a constant diameter along an entire length of the elongate shaft, may be about 0.0310±0.002 inches, for example.

The support catheters may be adapted to be compatible with particular guidewire sizes. For example, the large support catheter may have a lumen diameter of about 0.037 inches to be adapted to accommodate a 0.035-inch guidewire therethrough. The medium support catheter may have a lumen diameter of about 0.019 inches to be adapted to accommodate a 0.018-inch guidewire therethrough, while the small support catheter may have a lumen diameter of about 0.015 inches to be adapted to accommodate a 0.014-inch guidewire therethrough. Accordingly, and in some cases, the large support catheter may have an overall inner diameter (ID) of at least 0.035 inches, such as an ID of 0.037 inches, or more or 0.0385 inches or more. The medium support catheter may have an overall ID of at least 0.018 inches, such as an ID of 0.019 inches or more, or 0.0205 inches or more. The small support catheter may have an overall ID of at least 0.014 inches, such as an ID of 0.015 inches or more, or 0.0165 inches or more.

The support catheter 10 may have a variety of different lengths. For example, the large support catheter (adapted to work with an 0.035-inch guidewire) may have a length of 150 centimeters (cm), a length of 135 cm, a length of 90 cm or a length of 65 cm. The medium support catheter (adapted to work with an 0.018-inch guidewire) may have a length of 150 cm, a length of 135 cm or a length of 90 cm. The small support catheter (adapted to work with an 0.014-inch guidewire) may have a length of 150 cm, a length of 135 cm or a length of 90 cm. The length may be measured from the distal-most point on the distal tip 30 to a position proximate where the elongate shaft 12 meets the strain relief 20. These are just examples.

The wall thickness of the elongate shaft 12 of the large support catheter, which may be a constant thickness along an entire length of the elongate shaft, may be about 0.00825±0.0002 inches, for example. The wall thickness of the elongate shaft 12 of the medium support catheter, which may be a constant thickness along an entire length of the elongate shaft, may be about 0.00675±0.0002 inches, for example. The wall thickness of the elongate shaft 12 of the small support catheter, which may be a constant thickness along an entire length of the elongate shaft, may be about 0.0075±0.0002 inches, for example.

The elongate shaft 12 includes an inner polymeric layer 44 that defines a lumen 46 extending therethrough. It will be appreciated that the lumen 46 may be coaxially aligned with the lumen 24 that extends through the catheter hub 18 and thus allows devices such as but not limited to the guidewire 26, to extend all the way through the support catheter 10. This also means that fluids may be provided through the support catheter 10, if desired, by attaching an appropriate fluid source to the Luer fitting 22. In some cases, the inner polymer layer 44 is a bi-layer structure, including a first layer 48 and a second layer 50 surrounding the first layer 48. In some instances, the first layer 48 may be formed of a lubricious material such as a perfluoro material. As an example, the first layer 48 may be formed of PTFE (polytetrafluoroethylene). The second layer 50 may be formed of any suitable polymer, and in some cases may function as a tie layer between the first layer 48 and the polymer(s) within the outer layer 54. In some cases, the second layer 50 may be formed of a polyether block amide (PEBA), such as PEBAX 72D. In some cases, the second layer 50 may be formed of a similar material to that of the outer layer 53, such as a blend of VESTAMID® CARE ME71 and VESTAMID® CARE ML18, as discussed below. The first layer 48 may have an inner diameter that ranges from 0.036 to 0.040 inches for the large support catheter that ranges from 0.018 to 0.022 inches for the medium support catheter, and ranges from 0.015 to 0.018 inches for the small support catheter, respectively. The second layer 50 may have an outer diameter of about 0.0545±0.0007 inches for the large support catheter, about 0.0335±0.0005 inches for the medium support catheter, and about 0.0310±0.0007 inches, for the small support catheter, respectively.

The elongate shaft 12 includes a braided layer 52 that is disposed over the inner polymeric layer 44. In some cases, the filaments of the braided layer 52 are formed of a stainless-steel material. As an example, the braided layer 52 may be formed of 304V stainless steel. The braided layer 52 may be formed of one or more flat ribbons that may be braided in a 1-by-1 manner, or a 2-by-2 manner, or a 3-by-3 manner, or a 4-by-4 manner. The braided layer 52 may be formed of a flat stainless-steel ribbon having a thickness ranging from 0.0007 inches to 0.001 inches and a width ranging from 0.003 inches to 0.005 inches. In an example, the braided layer 52 may be formed of 304V stainless steel ribbons having a thickness of 0.0007±0.0002 inches and a width of 0.005±0.0005 inches. In other instances, the filaments of the braided layer 52 may have a circular cross-section. The braided layer may be formed of any number of flat ribbons, such as 32 ribbons, 16 extending in each helical direction. The braided layer may have a pick count of about 60 to about 80, or about 65 to about 80, about 70 to about 80, or about 75, for example. The pic count may be constant along an entire length of the braided layer, which extends along an entire length of the elongate shaft 12 from the proximal end of the elongate shaft 12 to the distal end of the elongate shaft 12 at the butt joint 32. In some cases, the 304V stainless steel ribbon may have a break load that is in a range of 0.942 pounds (lbs.) to 1.295 lbs. This is just an example, as other stainless-steel ribbons may be used.

The elongate shaft 12 includes an outer polymeric layer 54. In some instances, the outer polymeric layer 54 may be a blend of two or more different polymers. In some cases, the outer polymeric layer 54 may be a blend of two or more polyether block amide (PEBA) polymers. The outer polymeric layer 54 may be extruded directly onto the braided layer 52. In some instances, the outer polymeric layer 54 may be a continuous extrusion extending along an entire length of the elongate shaft 12, from the hub assembly 18 to the butt joint 32 proximate the distal tip 30. The polymer material and/or blend composition of the outer polymeric layer 54 may be uniform along the entire length of the outer polymeric layer 54 from the hub assembly 18 to the butt joint 32 proximate the distal tip 30. In some cases, this helps to embed the outer polymeric layer 54 at least partially into the braided layer 52, thereby improving the performance of the support catheter 10. In some cases, extruding the outer polymeric layer 54 directly onto the braided layer 52 may aid in partially embedding the braided layer 52 into the inner polymeric layer 44 (e.g., the tie layer 50 of the inner polymeric layer 44. In some cases, extruding the outer polymeric layer 54 directly onto the braided layer 52 may aid in bonding portions of the outer polymeric layer 54 flowing through gaps between the ribbons of the braided layer 52 directly to the tie layer 50 of the inner polymeric layer 44.

In some cases, suitable PEBA polymers are available commercially from Evonik Operations GmbH under the VESTAMID® CARE name. As an example, the outer polymeric layer 54 may be a blend of VESTAMID® CARE ME71 and VESTAMID® CARE ML18. These materials may be selected in accordance with their relative stiffness, for example. In some cases, the addition of the VESTAMID® CARE ML 18 to the polymer blend helps to provide stiffness while the VESTAMID® CARE ME71 helps with flexibility.

In some cases, the outer polymeric layer 54 may have a first blend of PEBA polymers for inclusion in the large support catheter. The outer polymeric layer 54 may have a second blend of PEBA polymers for inclusion in the medium support catheter, for example. In some cases, the outer polymeric layer 54 may have a third blend of PEBA polymers for inclusion in the small support catheter. In some cases, a first blend, a second blend and a third blend may individually refer to a combination of the same PEBA polymers, but with the PEBA polymers in differing relative concentrations. In some cases, a first blend, a second blend and a third blend may refer to combinations of the same PEBA polymers, in the same or differing relative concentrations, with changes in relative additives used in each of the blends. In some cases, a first blend, a second blend and a third blend may individually refer to combinations of different PEBA polymers forming the outer polymeric layer 54.

In some cases, the outer polymeric layer 54 may be formed of a blend that is predominantly VESTAMID® CARE ME71, with the following composition, particularly for the large support catheter (adapted to work with an 0.035-inch guidewire):

| Component | CAS # | Percent of Composition |
|---|---|---|
| VESTAMID ® CARE ME71 | n/a | 67.00 ± 2.00% |
| VESTAMID ® CARE ML18 | n/a | 19.37 ± 2.00% |
| Blanc Fixe XR-HN | 7727-43-7 | 9.00 ± 2.00% |
| CSRM045 Pigment Blue 29 | 101357-30-6/ | 0.52 ± 0.10% |
| | 57455-37-5 | |
| CSRM092 Pigment Red 122 | 980-26-7 | 0.60 ± 0.12% |
| Pigment White 6 | 13463-67-7 | 3.51 ± 0.70% |

In some cases, the outer polymeric layer 54 may be formed of blend that is predominantly VESTAMID® CARE ML18, with the following composition, particularly for the medium support catheter (adapted to work with an 0.018-inch guidewire) and the small support catheter (adapted to work with an 0.014-inch guidewire):

| Component | CAS # | Percent of Composition |
|---|---|---|
| VESTAMID ® CARE ME71 | n/a | 27.79 ± 1.00% |
| VESTAMID ® CARE ML18 | n/a | 58.58 ± 1.00% |
| Blanc Fixe XR-HN | 7727-43-7 | 9.00 ± 1.00% |
| CSRM045 Pigment Blue 29 | 101357-30-6/ | 0.52 ± 0.10% |
| | 57455-37-5 | |
| CSRM092 Pigment Red 122 | 980-26-7 | 0.60 ± 0.12% |
| Pigment White 6 | 13463-67-7 | 3.51 ± 0.70% |

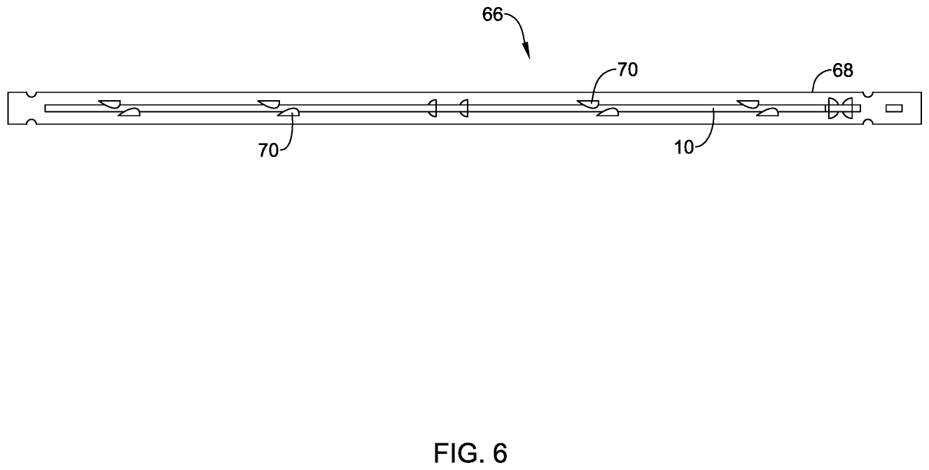

FIG. 5 provides further details regarding the braided layer 52. The braided layer 52 includes a total of thirty-two filaments, with sixteen filaments extending in a first helical direction and sixteen filaments extending in a second helical direction opposite the first helical direction. As can be seen, the individual filaments are wound in a two-by-two fashion. To illustrate, look at a first filament 56 and a second filament 58, each extending in a first helical direction, and a third filament 60 and a fourth filament 62, each extending in a second helical direction. The first filament 56 crosses over both the third filament 60 and the fourth filament 62, before passing under the next two crossing filaments (not labeled). The second filament 58 passes under the third filament 60 (and the preceding crossing filament) before passing over the fourth filament 62 (and the following crossing filament).

In some cases, the individual filaments forming the braided layer 52 may be arranged in a steep (45-degree angle relative to a longitudinal axis 64 of the braided layer 52) for the large support catheter. This angle is demonstrated with respect to the fourth filament 62, which can be seen as forming an angle α (alpha) relative to the longitudinal axis 64. The individual filaments forming the braided layer 52 may be arranged in a shallow (20-degree angle relative to the longitudinal axis 64 of the support catheter 10) for the large support catheter. In some cases, the individual filaments forming the braided layer 52 may be arranged in a steep (40-degree angle relative to the longitudinal axis 64 of the support catheter 10) for both the medium support catheter and the small support catheter. The braided layer 52 terminates at the butt joint 32 between the elongate shaft 12 and the distal tip 30, and thus the distal tip is free of any braided structure.

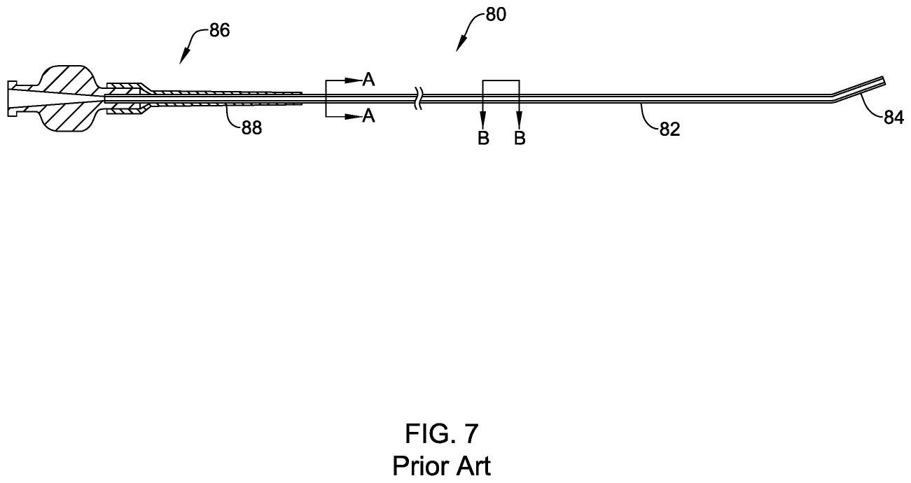

FIG. 6 is a schematic view of an assembly 66 that includes the support catheter 10 secured within a packaging structure 68. The packaging structure 68 includes a number of tabs 70 that serve to hold the support catheter 10 relative to the packaging structure 68. While not shown, it will be appreciated that the packaging structure 68 (and the support catheter 10) will be encapsulated within a material that maintains sterility of the support catheter 10 within the material, yet allows the support catheter 10 to be sterilized within the material. In some cases, the support catheter 10 may be sterilized by subjecting the packaging structure 68 (and the support catheter 10 within the packaging structure 68) to Ethylene Oxide sterilization, for example, although other sterilization processes are contemplated.

The packaging structure 68 is configured to maintain the support catheter 10 in a linear configuration. It is noted, that in instances in which the support catheter 10 includes a bend distal region, the bent distal region will be maintained in the packaging, while the remainder of the length of the elongate shaft will be held in a linear configuration in the packaging structure 68. While many elongate structures such as guidewires and catheters may be packaged with at least part of the elongate structure coiled up to preserve space, it has been found that in some cases storing the elongate structure in a coiled manner may impart a set to the elongate structure. Accordingly, maintaining the support catheter 10 in a straight configuration can help to eliminate any such set being imparted to any component of the support catheter 10. As a result, the support catheter 10 is better able to meet its desired pushability and torqueability performance characteristics.

### Experimental Section

Several sizes of the support catheter 10 were compared with similar-sized support catheters currently available on the market. The large (0.035-inch OD) support catheter was compared with a similar-sized support catheter available under the Navicross name having an angled tip, a length of 150 cm and an overall OD of 0.035 inches. The medium (0.018-inch OD) support catheter was compared with a similar-sized support catheter available under the CXI name having an angled tip, a length of 150 cm and an overall diameter of 0.018 inches. The small (0.014-inch OD) support catheter was compared with a similar-sized support catheter available under the CXI name. Each of the large, medium and small support catheters were constructed as shown and described with respect to FIGS. 1 to 6.

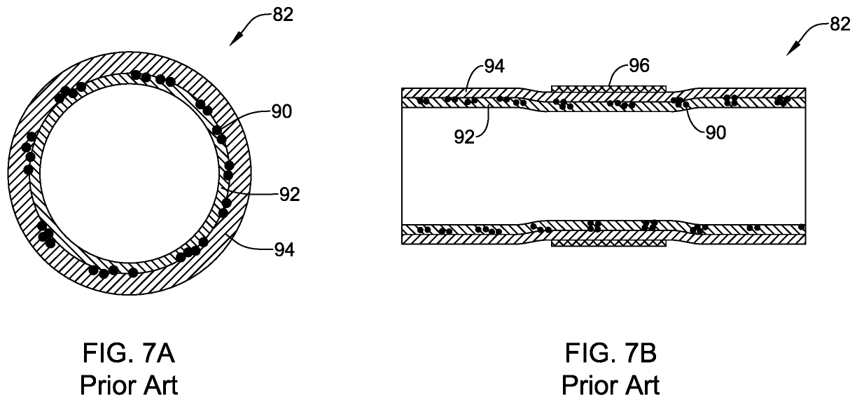

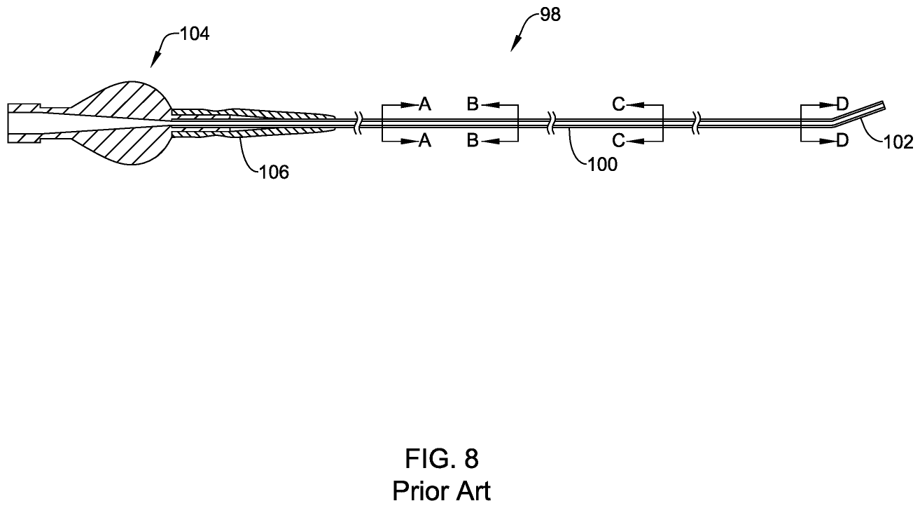

The Navicross device is illustrated in FIG. 7, which is a schematic side view of a device 80. FIG. 7A is a cross-sectional view while FIG. 7B is a longitudinal cross-sectional view. The Navicross device 80 includes an elongate shaft 82 extending from an angled distal tip 84 to a catheter hub 86 including a strain relief 88. As seen in FIG. 7A, the elongate shaft 82 includes a braid layer 90 that is disposed between an inner layer 92 and an outer layer 94. As seen in FIG. 7B, the braid layer 90 is at least partially embedded within the inner layer 92. A marker band 96 is at least partially embedded into the outer layer 94. The table below provides additional details regarding the Navicross device 80:

| | | Proximal | Transition | Distal |
|---|---|---|---|---|
| | Material | WT-AA | WT-BB | WT-CC |
| Outer Layer | Arnitel/Polyzen Blend | 0.00502 | | |
| Proximal | | | | |
| Outer Layer | Arnitel/Polyzen Blend | | 0.00435 | 0.00473 |
| Distal | | | | |
| Distal Marker | Platinum | | | |
| Braid | Stainless Steel | | | |
| Inner Layer | Arnitel/Polyzen Blend | 0.00188 | 0.00252 | 0.00237 |
| Total Wall | | 0.0069 | 0.00687 | 0.0071 |
| Thickness | | | | |
| I.D. | | 0.04136 | 0.0417 | 0.0414 |
| O.D. | | 0.05516 | 0.05544 | 0.05560 |
| Average Wall | 0.0070 | | | |
| Std Dev | 0.0001 | | | |

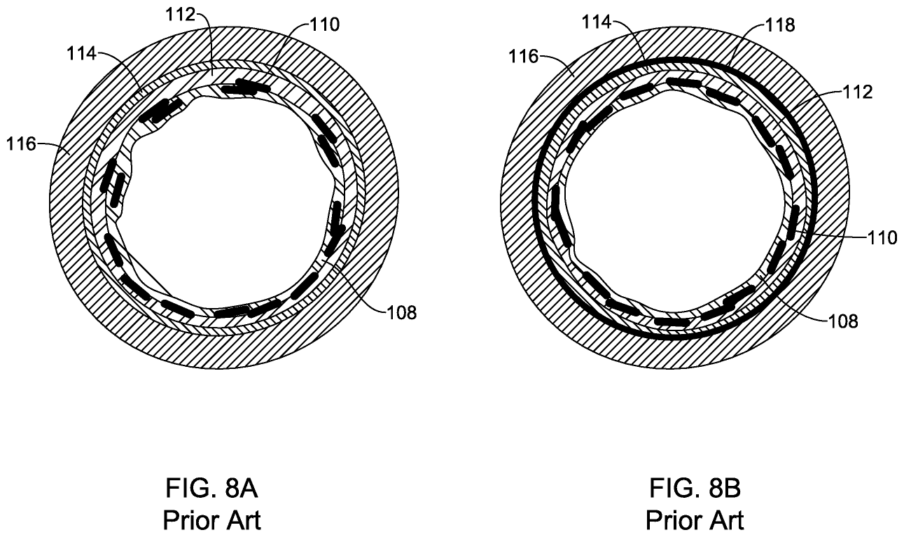

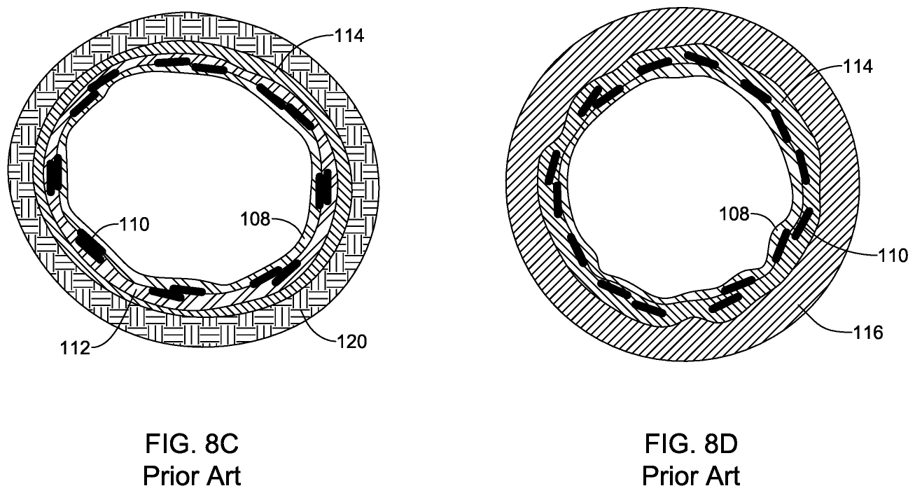

The CXI device is illustrated in FIG. 8, which a schematic side view of a device 98. FIGS. 8A, 8B, 8C and 8D are cross-sectional views taken at various points along the device 98. The device 98 includes an elongate shaft 100 extending from an angled distal tip 102 to a catheter hub 104 including a strain relief 106. As seen in the cross-sectional views, the elongate shaft 100 includes an inner layer 108, a braid layer 110 that is at least partially embedded into the inner layer 108, another layer 112 exterior to the inner layer 108, another layer 113 exterior to the layer 112, and an outer layer 116 that is exterior to the layer 114. FIG. 8B in particular shows a marker band 118 while FIG. 8C in particular shows a marker band layer 120. The table below provides additional details regarding the 0.018-inch OD size of the CXI device [:

| | | Proximal | | | Distal | | |
|---|---|---|---|---|---|---|---|
| | Material | WT-AA | WT-BB | WT-CC | WT-DD | WT-EE | WT-FF |
| Outer Layer Proximal | 72D Pebax | 0.00338 | 0.00331 | | | | |
| Outer Layer Distal | 54D Pebax | | | | 0.00343 | 0.00321 | 0.00348 |
| Proximal Marker layer | PU | | 0.00037 | | | | |
| Distal Marker | PU | | | 0.00351 | | | |
| 2nd Layer Proximal | Polyamide imide | 0.00092 | 0.00082 | 0.00103 | | | |
| 2nd Layer Distal | PU | | | | 0.00122 | 0.00213 | 0.00182 |
| 3rd Layer Proximal | ? | 0.00111 | 0.00125 | 0.00113 | | | |
| 3rd Layer Distal | Polyimide | | | | 0.00084 | | |
| Braid | Stainless Steel | | | | | | |
| Inner Layer | PTFE | 0.00125 | 0.00078 | 0.00088 | 0.00073 | 0.0007 | 0.00088 |
| Total Wall Thickness | | 0.00666 | 0.00653 | 0.00655 | 0.00622 | 0.00604 | 0.00618 |
| I.D. | | 0.0225 | 0.02218 | 0.0219 | 0.02232 | 0.02116 | 0.02146 |
| O.D. | | 0.03582 | 0.03524 | 0.035 | 0.03476 | 0.03324 | 0.03382 |
| Average Wall | 0.0064 | | | | | | |
| Std Dev | 0.0002 | | | | | | |

### Procedures

The procedure used for testing pushability is as follows. Pushability measures column stiffness, and is similar to a clinical scenario. The distal tip of the catheter is pushed against a load cell until a force of 50 grams is exerted. The slope of the force (measured at the load cell) and traveled distance of the catheter provides the measure of pushability. A higher slope means that the device has better column strength or pushability, and thus a higher result number is better than a lower result number.

The procedure used for testing torque response is as follows. The torque response test measures the transmission of torque from the proximal end of the device being tested to the distal end of the device being tested. The catheter (device) is placed inside a 2D torque fixture on an IDTE 3000, with the proximal hub attached to a proximal dynamic torque motor and the distal end being secured to a rotation out sensor. The proximal dynamic torque motor rotates the proximal hub for five (5) complete rotations (e.g., 1,800 degrees of rotation), and at the same time, the rotation out sensor measures output rotation at the distal end of the catheter shaft. The output of this test is the lag in torque, i.e., a delta difference in the rotational angle input (proximal end) to the rotational angle output (distal end). A lower result value is better than a higher result value. In other words, the output value from this test is the difference between the amount of rotational input at the proximal end of the catheter shaft minus the amount of rotation output at the distal end of the catheter shaft.

### Results

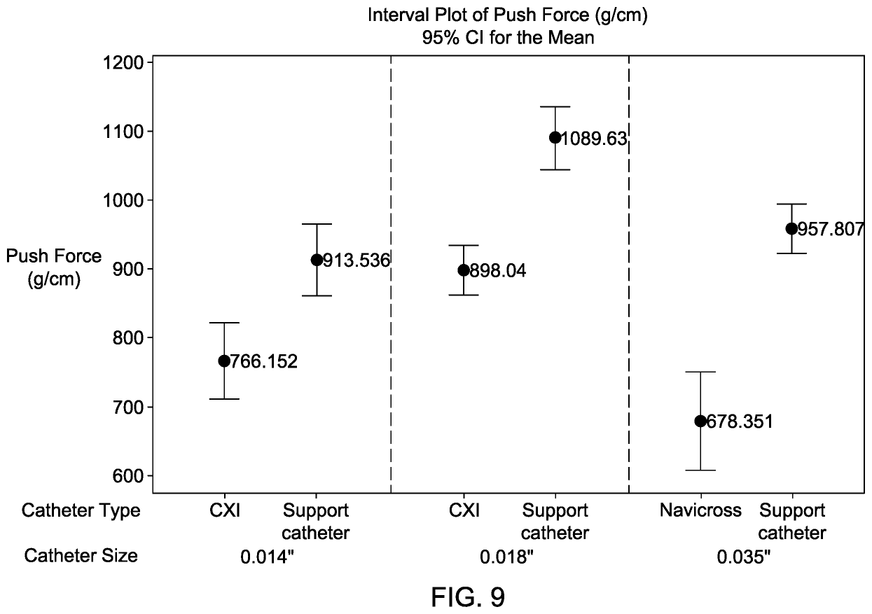

FIG. 9 is an interval plot of push force for the inventive support catheter, tested in the large (0.035-inch guidewire) size, the medium (0.018-inch guidewire) size and the small (0.014-inch guidewire) size, compared with the 0.014-inch OD CXI device, the OD CXI device and the 0.035-inch OD Navicross device. In each case, the inventive support catheter shows a substantially greater push force relative to the same sized competitive device. For the 0.014-inch size, the inventive support catheter had a push force of about 914 g/cm while the corresponding CXI device had a push force of only about 766 g/cm. The inventive support catheter had a push force that was more than 19 percent higher. For the 0.018-inch size, the inventive support catheter had a push force of about 1090 g/cm while the corresponding CXI device has a push force of only about 898 g/cm. The inventive support catheter had a push force that was more than 21 percent higher. For the 0.035-inch size, the inventive support concept had a push force of about 958 g/cm while the corresponding Navicross device had a push force of only about 678 g/cm. The inventive support catheter had a push force that was more than 41 percent higher.

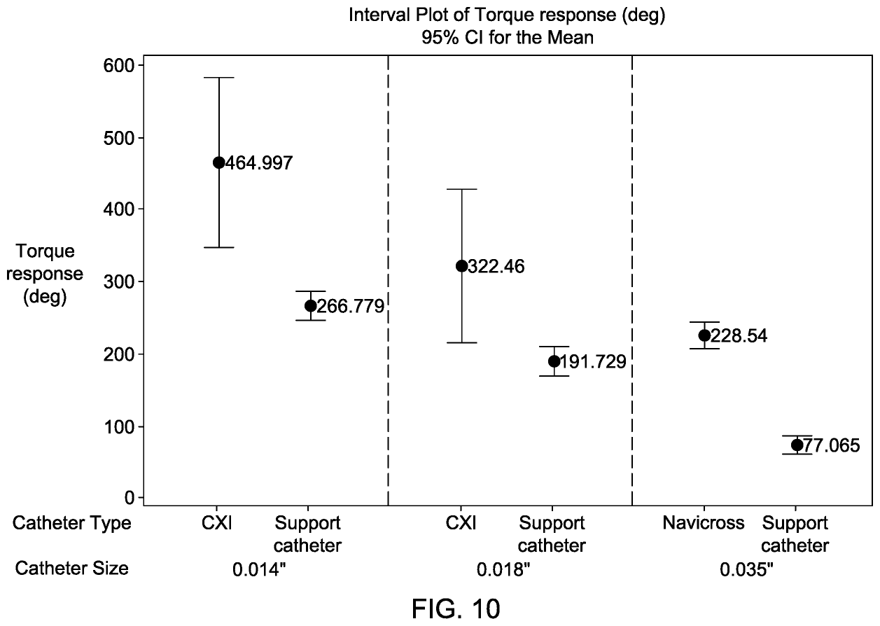

FIG. 10 is an interval plot of torque response for the inventive support catheter, tested in the large (0.035-inch guidewire) size, the medium (0.018-inch guidewire) size and the small (0.014-inch guidewire) size, compared with the 0.014-inch OD CXI device, the 0.018-inch OD CXI device and the 0.035-inch OD Navicross device. In each case, the inventive support catheter shows a substantially lower torque response relative to the same sized competitive device. For the 0.014-inch size, the inventive support catheter had a torque response of about 267 degrees while the corresponding CXI device has a torque response of about 465 degrees. For the 0.018-inch size, the inventive support catheter had a torque response of about 192 degrees while the corresponding CXI device has a torque response of about 322 degrees. For the 0.035-inch size, the inventive support catheter had a torque response of about 77 degrees while the corresponding Navicross device has a torque response of about 229 degrees.

The results show that the inventive support catheter, regardless of size, has a better pushability and a better torque response than the competitive products that they were tested against. The inventive support catheter clearly outperforms the competitive devices.

The devices described herein, and various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A support catheter comprising:
an elongate shaft extending from a proximal region to a distal region, the elongate shaft comprising:
a polymeric inner layer extending from the proximal region to the distal region;
a stainless-steel braided layer exterior to the polymeric inner layer and extending from the proximal region to the distal region; and
a polymeric blend outer layer partially embedded within the stainless-steel braided layer, the polymeric blend outer layer comprising a blend of polyether block amide (PEBA) polymers;
wherein the elongate shaft is adapted to provide a pushability value of at least 900 grams per centimeter and a torque response value of less than 300 degrees.

2. The support catheter of claim 1, further comprising a distal tip forming a butt joint with a distal end of the elongate shaft.

3. The support catheter of claim 2, wherein the braided layer extends along an entire length of the elongate shaft to the butt joint.

4. The support catheter of claim 2, further comprising:
a first radiopaque marker band located proximal of the butt joint; and
a polymeric sleeve spanning the butt joint;
wherein the polymeric sleeve extends over and surrounds the first radiopaque marker band and the distal tip.

5. The support catheter of claim 4, further comprising a second radiopaque marker band proximal of and spaced apart from the first radiopaque marker band, the outer layer reflowed around the second radiopaque marker band such that the second radiopaque marker band is embedded into the outer layer with the outer layer in contact with a proximal annular surface and a distal annular surface of the second radiopaque marker band.

6. The support catheter of claim 5, wherein an outer diameter of the second radiopaque marker band is greater than an outer diameter of the outer layer just proximal and distal of the second radiopaque marker band.

7. The support catheter of claim 1, wherein the polymeric blend outer layer comprises a blend of VESTAMID® Care ME71 and VESTAMID® Care ML 18.

8. The support catheter of claim 1, wherein the support catheter comprises one of a large support catheter adapted to accommodate a 0.035-inch guidewire, a medium support catheter adapted to accommodate a 0.018-inch guidewire, and a small support catheter adapted to accommodate a 0.014-inch guidewire, wherein the large support catheter is adapted to allow either one of the medium support catheters and the small support catheter to be advanced within the large support catheter.

9. The support catheter of claim 1, wherein the polymeric inner layer comprises a bi-layer structure with a perfluoro inner layer and a PEBA outer layer.

10. A support catheter adapted for use with a guidewire in order to provide additional support to the guidewire, the support catheter comprising:
an elongate shaft having a proximal end and a distal end, the elongate shaft including:
a perfluoro inner layer extending continuously from the proximal end to the distal end;
a stainless-steel braided layer exterior to the perfluoro inner layer and extending continuously from the proximal end to the distal end; and
a polymeric outer layer extending continuously from the proximal end to the distal end, the polymeric outer layer surrounding and partially embedded within the stainless-steel braided layer, the polymeric outer layer comprising a blend of VESTAMID® Care PEBA polymers;
a distal tip joined to the elongate shaft at a butt joint at the distal end of the elongate shaft;
wherein the elongate shaft is adapted to provide a pushability value of at least 900 grams per centimeter and a torque response value of less than 300 degrees.

11. The support catheter of claim 10, further comprising a polymeric tie layer disposed between the perfluoro inner layer and the stainless-steel braided layer.

12. The support catheter of claim 10, further comprising:
a first radiopaque marker band located proximal of the butt joint; and
a polymeric sleeve spanning the butt joint;
wherein the polymeric sleeve extends over and surrounds the first radiopaque marker band and the distal tip.

13. The support catheter of claim 10, wherein the support catheter is packaged in a linear orientation in order to avoid providing a curved set to the support catheter.

14. The support catheter of claim 10, wherein the support catheter comprises one of a large support catheter adapted to accommodate a 0.035-inch guidewire, a medium support catheter adapted to accommodate a 0.018-inch guidewire, and a small support catheter adapted to accommodate a 0.014-inch guidewire.

15. The support catheter of claim 14, wherein the outer layer comprises a blend of VESTAMID® Care ME71 and VESTAMID® Care ML 18, and wherein:
the large support catheter has a polymeric blend outer layer comprising VESTAMID® Care ME71 and VESTAMID® Care ML 18 blended in a first ratio;
the medium support catheter has a polymeric blend outer layer comprising VESTAMID® Care ME71 and VESTAMID® Care ML 18 blended in a second ratio; and
the small support catheter has a polymeric blend outer layer comprising VESTAMID® Care ME71 and VESTAMID® Care ML 18 blended in a third ratio.

16. The support catheter of claim 14, wherein the large support catheter is adapted to allow either one of the medium support catheter and the small support catheter to be advanced within the large support catheter.

17. A support catheter, the support catheter comprising:
an elongate shaft extending from a proximal end to a distal end, the elongate shaft including:
a polymeric inner layer extending continuously from the proximal end to the distal end;
a braided layer surrounding the polymeric inner layer and extending continuously from the proximal end to the distal end; and
a polymeric blend outer layer extending continuously from the proximal end to the distal end, the polymeric blend outer layer surrounding and partially embedded within the braided layer, the polymeric blend outer layer comprising a blend of polyether block amide (PEBA) polymers;
a distal tip secured to the distal end of the elongate shaft via a butt joint; and
a plurality of radiopaque marker bands disposed within the distal region proximal of the butt joint;
wherein the support catheter is adapted to provide a pushability value of at least 900 grams per centimeter and a torque response value of less than 300 degrees.

18. The support catheter of claim 17, further comprising a polymeric sleeve spanning the butt joint;
wherein the polymeric sleeve extends over and surrounds a distalmost one of the plurality of radiopaque marker bands and the distal tip.

19. The support catheter of claim 18, wherein the plurality of radiopaque marker bands includes a proximalmost one of the plurality of radiopaque marker bands located proximal of the distalmost one of the plurality of radiopaque marker bands, wherein the polymeric blend outer layer is reflowed around the proximalmost one of the plurality of radiopaque marker bands such that the proximalmost one of the plurality of radiopaque marker bands is embedded into the polymeric blend outer layer with the polymeric blend outer layer in contact with a proximal annular surface and a distal annular surface of the proximalmost one of the plurality of radiopaque marker bands.

20. The support catheter of claim 17, wherein the polymeric blend outer layer comprises a blend of VESTAMID® Care ME71 and VESTAMID® Care ML 18.
